(19) United States
(12) Patent Application Publication
(10) Pub. No.: US 2008/0141683 A1
(43) Pub. Date: Jun. 19, 2008

(54) Chambered instant cold pack

(76) Inventors: Amanda L. O'Connor, Appleton, WI (US); Marcy E. Sojka, Neenah, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/638,767

(22) Filed: Dec. 13, 2006

(51) Int. Cl. F25D 5/00; A61F 7/00

(52) U.S. Cl. 62/4; 607/96

(57) ABSTRACT

Cold therapy is an established practice used in the medical profession to treat certain limb injuries, such as sprained or strained arm or leg muscles or injuries to joints. Generally, these types of injuries should be chilled to slow blood flow, which reduces swelling, pain, and further damage. Cold therapy is also an established practice used to treat migraine and other types of headaches. A typical course of cold therapy treatment is to apply ice for a specified period to the injured region of a limb, the neck, face, or head. Alternatively, a pack or bag containing a chemical agent that reacts (endothermically) to produce cold may be applied to the injured region, the neck, face, or head.

### BACKGROUND OF THE INVENTION

Cold therapy is an established practice used in the medical profession to treat certain limb injuries, such as sprained or strained arm or leg muscles or injuries to joints. Generally, these types of injuries should be chilled to slow blood flow, which reduces swelling, pain, and further damage. Cold therapy is also an established practice used to treat migraine and other types of headaches. A typical course of cold therapy treatment is to apply ice for a specified period to the injured region of a limb, the neck, face, or head. Alternatively, a pack or bag containing a chemical agent that reacts (endothermically) to produce cold may be applied to the injured region, the neck, face, or head.

A number of devices that use endothermic reactions for cooling body parts are known. For example, U.S. Pat. No. 4,986,076 to Kirk et al. and U.S. Pat. No. 2,898,744 to Robbins both disclose a flexible, plastic cooling bag sealed along its edges. The cooling bag is separated by a frangible barrier into two portions: a freezing chemical mixture (salt) portion and a liquid (water) portion. A cooling reaction is activated by squeezing or applying pressure to the bag, which ruptures the frangible barrier and thus allows the salt and liquid portions to mix. The resulting chemical mixture causes an endothermic reaction, which produces a cooling effect. The cooled bag is applied to a body part.

A concern with such endothermic cold packs is that the solution that is formed ends up at the bottom (i.e., the lowest point) of the enclosure. The localization of the solution within the enclosure results in temperature differences within the cold pack. The temperature differences that are generated within the cold pack cause the cold pack to cool various sections of an area at differing rates.

Accordingly, there is a need for a cold pack that uniformly cools an area of the body. The cold pack should also be readily portable and adaptable for various needs of users.

### SUMMARY OF THE INVENTION

The present inventors undertook intensive research and development efforts concerning improving instant cold therapy. The present invention is directed in part to a cold pack including an enclosure, a solute within the enclosure, a liquid within the enclosure, and a membrane segregating the liquid from the solute. Further, rupturing the membrane mixes the liquid with the solute to produce an endothermic solution within the enclosure. In addition, the enclosure includes a first compartment, a second compartment, and a connection between the first compartment and the second compartment. The connection is adapted to pass the endothermic solution between the first and the second compartment.

Another aspect of the present invention is directed to a cold pack including an enclosure, a solute within the enclosure, a liquid within the enclosure, and a membrane segregating the liquid from the solute. Further, rupturing the membrane mixes the liquid with the solute to produce an endothermic solution within the enclosure. In addition, the enclosure includes a first compartment, a second compartment, and a connection between the first compartment and the second compartment. The connection is adapted to pass the endothermic solution between the first and the second compartment. Further, a force other than gravity is needed for the endothermic solution to pass through the connection between the first compartment and the second compartment. Additionally, the cold pack includes a mechanism for temporarily blocking the connection between the first compartment and the second compartment to prevent the endothermic solution from passing between the first compartment and the second compartment.

A third aspect of the present invention is directed to a cold pack including an enclosure, a solute within the enclosure, a liquid within the enclosure, and a membrane segregating the liquid from the solute. The liquid may be water. The solute may be ammonium nitrate, and the membrane may be polyethylene. Further, rupturing the membrane mixes the liquid with the solute to produce an endothermic solution within the enclosure. In addition, the enclosure includes a first compartment, a second compartment, and a connection between the first compartment and the second compartment. The connection is adapted to pass the endothermic solution between the first and the second compartment.

### BRIEF DESCRIPTION OF THE DRAWINGS

The present invention will be more fully understood, and further features will become apparent, when reference is made to the following detailed description and the accompanying drawings. The drawings are merely representative and are not intended to limit the scope of the claims. Like parts depicted in the drawings are referred to by the same reference numerals.

FIG. 1 illustrates a schematic section view of a cold pack.

FIG. 2 illustrates a schematic section view of the cold pack shown in FIG. 1 with a membrane ruptured within the cold pack to produce an endothermic solution within the cold pack.

FIG. 3 illustrates a schematic section view of another cold pack.

FIG. 4 illustrates a schematic section view of the cold pack shown in FIG. 3 with a membrane ruptured within the cold pack to produce an endothermic solution within the cold pack.

FIG. 5A illustrates a plan view of yet another cold pack with multiple compartments.

FIG. 5B illustrates a plan view of the cold pack shown in FIG. 5A in a first orientation with a membrane ruptured within the cold pack to produce an endothermic solution where the endothermic solution is distributed in one compartment within the cold pack.

FIG. 5C illustrates a plan view of the cold pack shown in FIG. 5B in a second orientation with a membrane ruptured within the cold pack to produce an endothermic solution.

FIG. 5D illustrates a plan view of the cold pack shown in FIG. 5A in a first orientation with a membrane ruptured within the cold pack to produce an endothermic solution where the endothermic solution is distributed in more than one compartment within the cold pack.

FIGS. 6A-6D illustrate several different cold packs with enclosures having compartments and connections between the compartments.

### DETAILED DESCRIPTION OF THE INVENTION

In the following detailed description, reference is made to the accompanying drawings which show specific embodiments in which the invention may be practiced. These embodiments are described in sufficient detail to enable those skilled in the art to practice the invention. It is to be understood that other embodiments may be utilized and structural changes made, such that the following detailed description is not to be taken in a limiting sense.

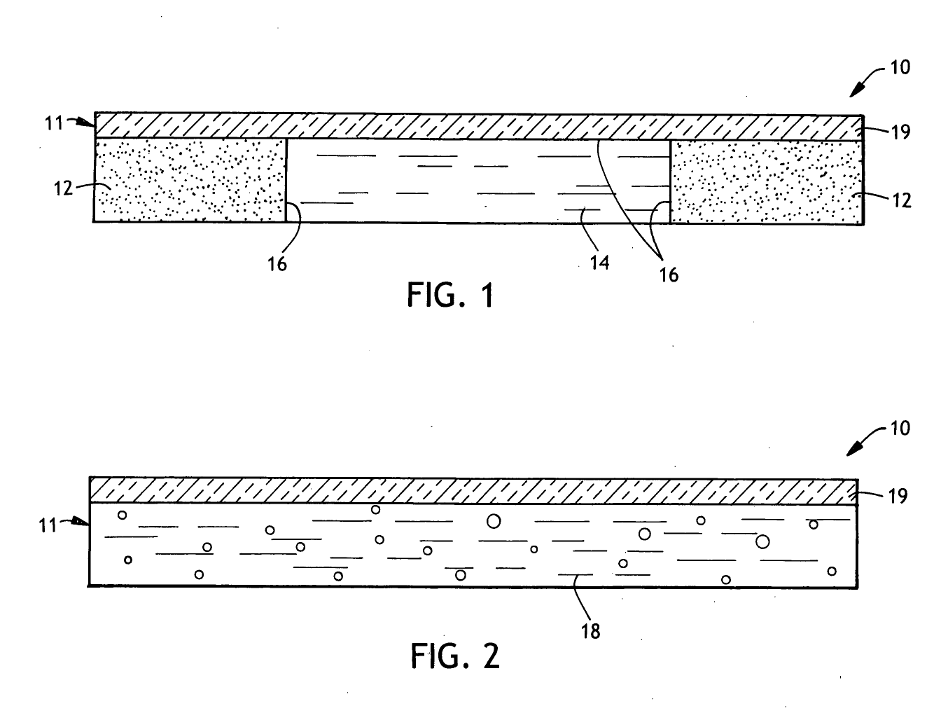

FIGS. 1 and 2 illustrate a cold pack 10. The cold pack 10 includes an enclosure 11 with a solute 12 and a liquid 14 sealed inside the enclosure 11. The solute 12 and the liquid 14 are segregated within the enclosure 11 by a membrane 16 (see FIG. 1). Rupturing the membrane 16, for example by applying pressure to the enclosure 11, mixes the liquid 14 (e.g., water) with the solute 12 to produce an endothermic solution 18 within the enclosure 11 (see FIG. 2). Substantially all of the solute 12 rapidly dissolves within the liquid 14 such that the cold pack 10 quickly reaches its cooling temperature.

Cold pack 10 may also include an insulation layer 19 that insulates a portion of the enclosure 11 from the surrounding environment. It should be noted that insulation layer 19 may have any size or shape and may be in or on the enclosure 11. Insulation layer 19 should be positioned on a side of enclosure 11 that is opposite to the side of enclosure 11 which is to be located on, or near, the body. The insulation layer 19 then serves to reduce warming of the cold pack 10 by the ambient environment without inhibiting heat transfer from the body to the cold pack 10.

Membrane 16 may be polyethylene (among other materials). In addition, any conventional solutes may be used to induce an endothermic reaction within cold pack 10. One example solute includes ammonium nitrate. The rate at which the solute dissolves into the liquid, and thus the rate of cooling, may be affected by the particle size of the solute. For example, if a rapid dissolution is desired, the pieces that form the solute 12 may be between about 0.001 and 0.025 inches, although it should be noted that smaller pieces may be used and some small minority of pieces may be larger than 0.025 inches.

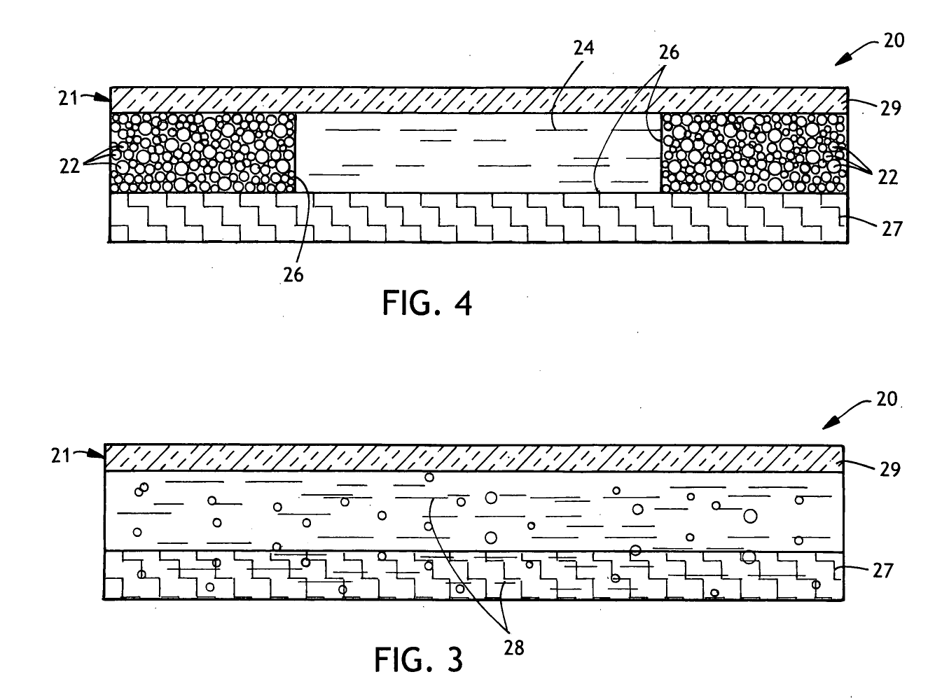

FIGS. 3 and 4 illustrate a cold pack 10. The cold pack 10 includes an enclosure 11 and a liquid 14 and a solute 12 sealed inside the enclosure 11. The solute 12 and the liquid 14 (e.g., water) are segregated within the enclosure 11 by a membrane 16.

The cold pack 10 further includes an absorbent core, such as absorbent layer 27, within the enclosure 11 (see FIG. 3). The absorbent layer 17 retains an endothermic solution 18 that is formed within the enclosure 11 by rupturing the membrane 16 (see FIG. 4). Once the solute 12 and the liquid 14 are mixed to form the endothermic solution 18, the absorbent layer 17 spreads the endothermic solution 18 throughout the enclosure 11 such that the cold pack 10 uniformly cools an injured portion of a body when the cold pack 10 is positioned on, or near, the body.

The membrane 16 may also isolate the absorbent layer 17 from the solute 12 and/or the liquid 14 until the membrane 16 is ruptured to mix the solute 12 and the liquid 14. In some sample forms, the solute 12 may be interspersed with the absorbent layer 17 before membrane 16 is ruptured. The absorbent core may take forms other than absorbent layer 17 and may be pulp fiber (among other materials).

It should be noted that in the example cold pack 10 illustrated in FIGS. 3 and 4, solute 12 may be in pellet or powder form. In addition, cold pack 10 may include an insulation layer 19 which is similar to insulation layer 19 described above with regard to FIGS. 1 and 2.

In some example embodiments, the solute 12 may be integral with the absorbent layer 17 as opposed to being initially isolated from the absorbent layer 17. Although not specifically illustrated, these types of example embodiments may include an enclosure 11 and a membrane 16 that initially segregates a liquid 14 from a solute-filled absorbent layer 17 within the enclosure 11. Combining the solute 12 with the absorbent layer may simplify fabrication of such cold packs.

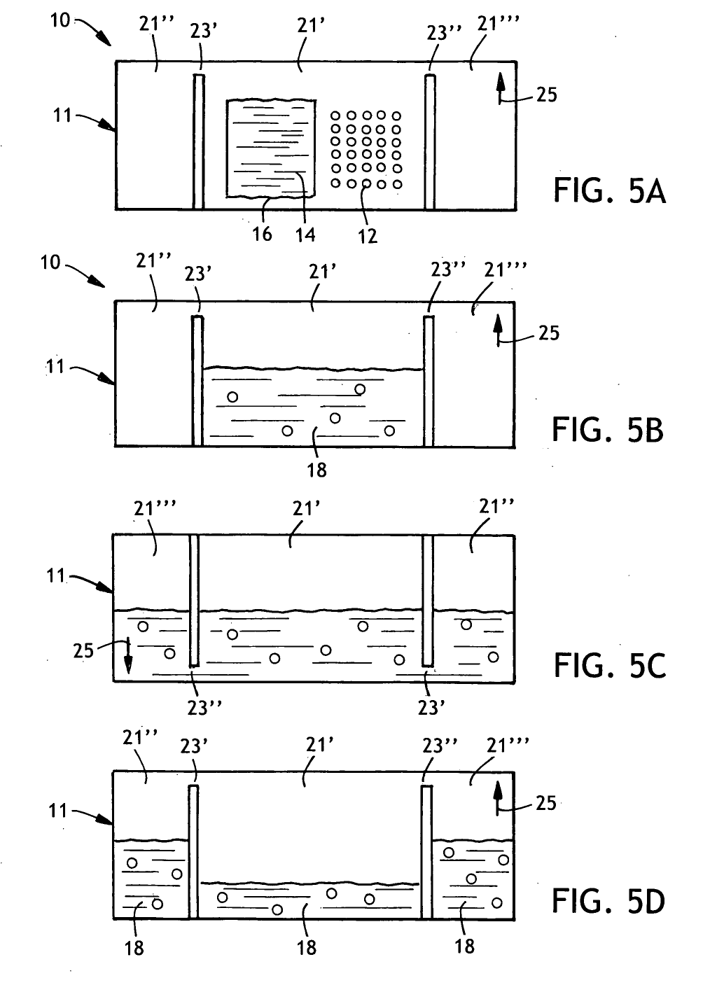

FIGS. 5A-5D illustrate a plan view of yet another cold pack 10 with multiple compartments 21′, 21″, 21′″ within the enclosure 11. The enclosure 11 also includes connections 23′, 23″ that are adapted to pass material between the compartments such as liquid 14, solute 12, or endothermic solution 18. The cold pack 10 illustrated in FIGS. 5A-5D includes a first compartment 21′, a second compartment 21″, and a third compartment 21′″. This cold pack 10 may be suitable for treating the forehead and temples. The first compartment 21′ may generally correspond with the area of the cold pack 10 that contacts the forehead. The second compartment 21″ and the third compartment 21′″ may generally correspond with areas of the cold pack 10 that contact the temples.

Sufferers of headaches, and in particular migraine headaches, may find it comforting to lie down, or be in a reclined position while applying cooling to their heads. Further, sufferers of headaches may also desire a uniform distribution across the temple and forehead area. The compartments 21′, 21″, 21′″, and connections 23′, 23″ allow for the endothermic solution 18 to move around the enclosure 11 under certain situations, and prevent movement of the endothermic solution 18 around the enclosure 11 under other situations. Specifically, with the cold pack 10 in a first orientation in relation to gravity as shown in FIGS. 5A, 5B, and 5D with indicator arrow 25 pointed in the upward direction, the connections 23′, 23″ are positioned near top of the enclosure 11, such that the endothermic solution 18 does not pass through the connections 23′, 23″. However, with the cold pack 10 in a second orientation in relation to gravity as show in FIG. 5C with indicator arrow 25 pointed downward, the connection 23′, 23″ are positioned near the bottom of the enclosure 11, such that the endothermic solution 18 does pass between the compartments 21′, 21″, and 21′″. A user may distribute the endothermic solution 18 between the first, second, and third compartments 21′, 21″, 21′″ in a manner that best suits their needs. For example, one sufferer may chose to have equal amounts of endothermic solution 18 in each of the compartments 21′, 21″, 21′″, while another sufferer may choose to have a disproportionate amount of endothermic solution in the first (center) compartment 21′. Regardless, the compartments 21′, 21″, 21′″ and connections 23′, 23″ allow the sufferer the ability to counteract the natural distribution of liquid that gravity would affect on an open enclosure.

The cold pack 10 is illustrated in FIGS. 5A-5D in four distinct configurations. In the first configuration, illustrated in FIG. 5A, the membrane 16 is intact, and the liquid 14 and the solute 12 are separate. In the second configuration, illustrated in FIG. 5b, the membrane 16 has been ruptured and the solute 12 and the liquid 14 have formed an endothermic solution 18. Further in this second configuration, the endothermic solution 18 is contained exclusively within the first compartment 21′, and endothermic solution does not pass through the connection 23′, 23″. In the third configuration, illustrated in FIG. 5C, the cold pack has been oriented such that the endothermic solution 18 has passed through the connection 23′, 23″ into the second compartment 21″, and the third compartment 21′″. As illustrated in FIG. 5C, the endothermic solution 18 may be distributed proportionately in each compartment 21′, 21″, 21′″. In the fourth configuration, illustrated in FIG. 5D, the endothermic solution is distributed disproportionately in the compartment 21′, 21″, 21′″, specifically a proportionally larger amount of endothermic solution 18 is located in the second compartment 21″, and the third compartment 21′″ than is located in the first compartment 21′. Depending on the specific design and use of the cold pack 10, a user may choose to locate various amounts of endothermic solution 18 in the compartments 21′, 21″, 21′″.

Endothermic solution may pass between the compartments 21′, 21″, 21′″ under the force of gravity. Alternatively, the connection 23′, 23″ may be designed such that a force other than gravity is needed for the endothermic solution 18 to pass through the connection between the compartments 21′, 21″, 21′″. This force may be pressure applied to the exterior of the enclosure 11 increasing the pressure of the endothermic solution 18 in one of the compartments 21′, 21″, 21′″ in relation to another compartment 21′, 21″, 21′″.

The compartments 21′, 21″, 21′″ may be formed in any suitable manner. For example, the enclosure 11 may be formed from two planar sheets of material joined together along the periphery of the two sheets. The compartments 21′, 21″, 21′″ may be formed by joining portions of the two sheets forming the compartments 21′, 21″, 21′″. The sheets may be joined using thermal, ultrasonic, or adhesive bonding. Alternatively, two or three compartments 21′, 21″, 21 ′″ may be formed separately, and then joined with connections. For example, the connections may be straws or tubes which connect the compartments 21′, 21″, 21′″.

The connections 23′, 23″ may be adapted to remain open at all times. Alternatively, the connections 23′, 23″ may be adapted to be blocked either temporarily or permanently. For example, the material which surrounds the connection 23′, 23″ may be elastic such that by applying tension the connection 23′, 23″ opens and allows endothermic solution 18 to pass between the compartments 21′, 21″, 21′″. Further, upon release of the tension, the connections 23′, 23″ may become blocked, preventing the endothermic solution 18 from passing between the compartments 21′, 21″, 21′″. Many suitable devices may be used to temporarily block the connection 23′, 23″, for example, a valve, a clip, a tie, a plug, or a zipper seam may be used. As used herein, the term “zipper seam” refers to self-mating rib and flange seams such as are commonly used with sandwich bags. A suitable zipper seam is described in U.S. Pat. No. 6,544,604 issued Apr. 15, 2003 to Galkiewicz et al. Many suitable devices may be used to permanently block the connection 23′, 23′″; for example, an adhesive, a thermal seal, or a solvent weld.

Depending on the specific design and desired final use of the cold pack 10, the solute 12 and the liquid 14 may be located anywhere within the enclosure 11. For example the solute 12 may be located in the first compartment 21′ and the liquid 14 may be located in the second compartment 21″. Alternatively the solute 12 may be located in the first compartment 21′ and the liquid 14 may also be located in the first compartment 21′.

The first compartment 21′ and the second compartment 21″ may include absorbent core 17 adapted to retain the endothermic solution 18. For example the first compartment 21′ may include an absorbent core 17 while the second compartment 21″ may not include an absorbent core 17. This may be advantageous where the first compartment 21′ is adapted to be used such that it is elevated relative to the second compartment 21″ during use, for example where the first compartment 21′ is adapted to be used on the forehead and the second compartment 21″ is adapted to be used on a temple when the user is in a reclined position. In this configuration, the second compartment 21″ may or may not include a second absorbent core.

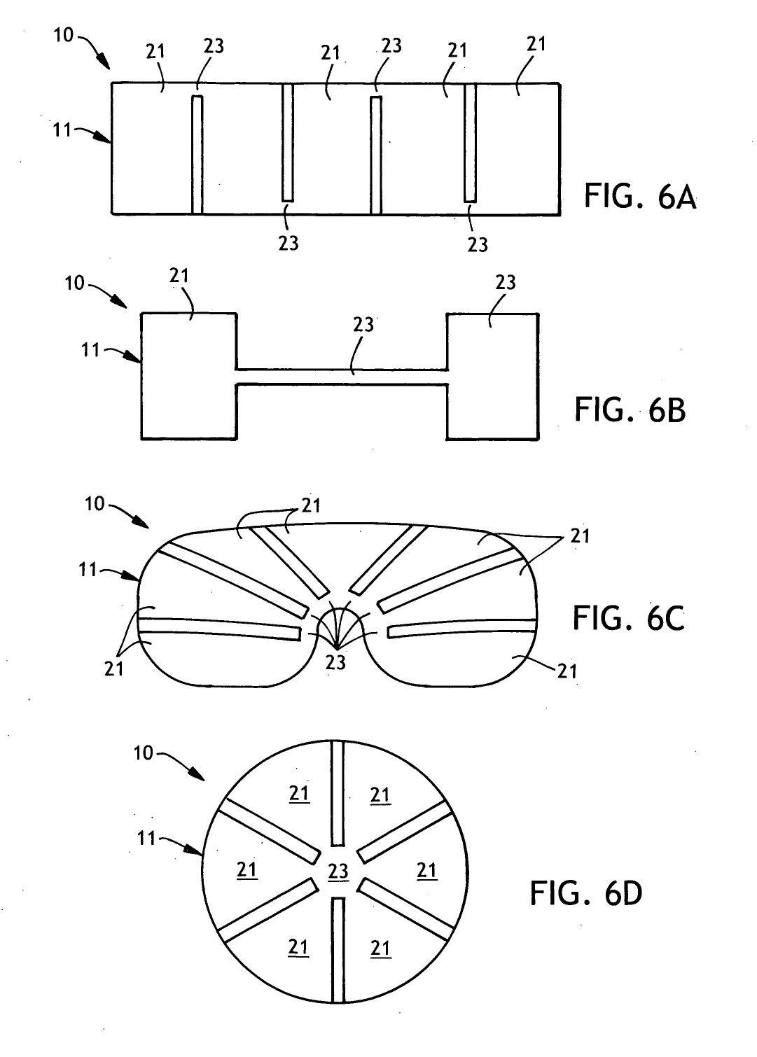

FIGS. 6A-6D illustrate several different cold packs with enclosures having compartments and connections between the compartments. FIG. 6A illustrates a cold pack 10 having five compartments 21, and four connections 23 between the compartments 21. As illustrated in FIG. 6A, the connections 23 are located at the top and bottom of the enclosure 11, alternating top to bottom from left to right. This cold pack 10 may be suitable for use in cooling an ankle or a neck.

FIG. 6B illustrates a cold pack 10 having two compartments 21 and one connection 23 between the compartments 21. As illustrated in FIG. 6B, the compartments 21 are located at opposite ends of a relatively long connection 23. This cold pack 10 may be suitable for use in cooling the left and right temple, or the left and right side of a knee, ankle, elbow, or shoulder.

FIG. 6C illustrates a cold pack 10 having seven compartments 21 and six connections 23 between the compartments 21. As illustrated in FIG. 6C, the cold pack 10 is designed and shaped to cover the eyes of a user, with a notch for the nose. The compartments 21 and connections 23 are designed such that a user may distribute endothermic solution such that a uniform cooling may occur around the eyes.

FIG. 6D illustrates a cold pack 10 having six compartments 21 and six connections 23 between the compartments 21. As illustrated in FIG. 6C, cold pack 10 is generally circular in shape and the compartments 21 have the shape of a sector of a circle of approximately 60 degrees. The connection 23 is located at the center of the circle. This cold pack 10 may be suitable for use in cooling the knee, elbow, shoulder, or ankle.

In alternative forms, the cold pack 10 may include numerous additional features. For example the cold pack 10 may include outer covers made from a wide variety of materials, including, for example, woven fabrics and nonwoven fabrics or webs. Nonwoven materials suitable for use with the present invention include, for example, a multilayer laminate such as a spunbond/meltblown/spunbond (“SMS”) material. An example of such a fabric is disclosed in U.S. Pat. No. 4,041,203 and is hereby incorporated by reference. Additional features may include material adapted to extending the cooling duration of the cold pack 10, for example as disclosed in U.S. Pat. No. 6,881,219 and hereby incorporated by reference.

In alternative forms, a release layer (not shown) may be detachably mounted to the cold pack using an adhesive. The release layer may be removed from the cold pack leaving only the adhesive on the cold pack. The remaining adhesive provides a means for directly or indirectly securing the cold pack to a body, flexible wrap, and/or other device.

A method of cooling a portion of a body is described herein with reference to FIGS. 5A-5D and FIGS. 6A-6D. In one form, the method includes segregating a solute 12 from a liquid 14 where the solute 12 and the liquid 14 are inside of a cold pack 10 (FIGS. 5A, 6A-6D). The method further includes mixing the solute 12 with the liquid 14 to form an endothermic solution 18 within the cold pack 10, distributing the endothermic solution 18 though connections 23 within the cold pack 10, and applying the cold pack 10 to the portion of the body. In some sample forms of the method, mixing the solute 12 and the liquid 14 to form an endothermic solution 18 includes rupturing a membrane 16 that segregates the solute 12 from the liquid 14 within the cold pack 10.

In some sample forms of the method, distributing the endothermic solution 18 throughout the cold pack 10 includes retaining the endothermic solution 18 within an absorbent core, such as absorbent layer 27. In some alternative forms, the method includes mixing the solute 12 and the liquid 14 within the absorbent layer 17. It should also be noted that the solute 12 may be in pellet form or powder form.

The size and shapes of the cold packs described herein will depend on the applications where the cold packs will be used (among other factors). In addition, the membranes within the enclosures may have any size, number, arrangement, and configuration as long as the membrane (i) segregates the solute from the liquid; and (ii) is capable of being ruptured so that the solute can be mixed with the liquid to form an endothermic solution.

The operations discussed above with respect to the described methods may be performed in a different order from those described herein. It should be noted that attaching a cold pack to a body includes attaching the cold pack directly or indirectly to the body. In addition, FIGS. 1-6D are representational and are not necessarily drawn to scale. Certain proportions thereof may be exaggerated, while others may be minimized.

While the invention has been described in detail with respect to the specific aspects thereof, it will be appreciated that those skilled in the art, upon attaining an understanding of the foregoing, may readily conceive of alterations to, variations of, and equivalents to these aspects which fall within the spirit and scope of the present invention, which should be assessed accordingly to those of the appended claims.

What is claimed is:

1. A cold pack comprising:
an enclosure;
a solute within the enclosure;
a liquid within the enclosure; and
a membrane segregating the liquid from the solute;
wherein rupturing the membrane mixes the liquid with the solute to produce an endothermic solution within the enclosure; and
the enclosure includes a first compartment, a second compartment, and a connection between the first compartment and the second compartment, the connection adapted to pass the endothermic solution between the first and the second compartment.

2. The cold pack of claim 1 having a first orientation in relation to gravity and a second orientation in relation to gravity, wherein the endothermic solution passes through the connection between the first compartment and the second compartment in the first orientation and the endothermic solution does not pass through the connection between the first compartment and the second compartment in the second orientation.

3. The cold pack of claim 1 wherein a force other than gravity is needed for the endothermic solution to pass through the connection between the first compartment and the second compartment.

4. The cold pack of claim 1 further comprising means for temporarily blocking the connection between the first compartment and the second compartment to prevent the endothermic solution from passing between the first compartment and the second compartment.

5. The cold pack of claim 4 wherein the means for temporarily blocking the connection is a valve.

6. The cold pack of claim 4 wherein the means for temporarily blocking the connection is a clip, a tie, or a zipper seam.

7. The cold pack of claim 1 further comprising means for permanently blocking the connection between the first compartment and the second compartment to prevent the endothermic solution from passing between the first compartment and the second compartment.

8. The cold pack of claim 7 wherein the means for permanently blocking the connection is an adhesive seal.

9. The cold pack of claim 1 wherein the liquid is water.

10. The cold pack of claim 1 wherein the solute is ammonium nitrate.

11. The cold pack of claim 1 wherein the membrane is polyethylene.

12. The cold pack of claim 1 wherein each piece of the solute is substantially between 0.001 and 0.025 inches.

13. The cold pack of claim 1 wherein the solute is located in the first compartment and the liquid is located in the second compartment.

14. The cold pack of claim 1 wherein the solute is located in the first compartment and the liquid is located in the first compartment.

15. The cold pack of claim 1 further comprising a first absorbent core within the first compartment, the absorbent core adapted to retain the endothermic solution in the first compartment.

16. The cold pack of claim 15 further comprising a second absorbent core within the second compartment, the second absorbent core adapted to retain the endothermic solution in the second compartment.

17. The cold pack of claim 15 wherein the membrane segregates the liquid from the first absorbent core.

18. The cold pack of claim 15 wherein the first absorbent core is an absorbent layer.

19. The cold pack of claim 17 wherein substantially all of said solute is dissolved in said liquid to form said endothermic solution before said endothermic solution is retained by said absorbent core.

20. A cold pack comprising:
an enclosure;
a solute within the enclosure;
a liquid within the enclosure; and
a membrane segregating the liquid from the solute;
wherein rupturing the membrane mixes the liquid with the solute to produce an endothermic solution within the enclosure;
the enclosure includes a first compartment, a second compartment, and a connection between the first compartment and the second compartment, the connection adapted to pass the endothermic solution between the first and the second compartment;
a force other than gravity is needed for the endothermic solution to pass through the connection between the first compartment and the second compartment; and.
the cold pack further comprising means for temporarily blocking the connection between the first compartment and the second compartment to prevent the endothermic solution from passing between the first compartment and the second compartment.

21. The cold pack of claim 20 wherein the means for temporarily blocking the connection is a valve.

22. The cold pack of claim 20 wherein the means for temporarily blocking the connection is a clip, a tie, or a zipper seam.

23. The cold pack of claim 20 further comprising means for permanently blocking the connection between the first compartment and the second compartment to prevent the endothermic solution from passing between the first compartment and the second compartment.

24. The cold pack of claim 23 wherein the means for permanently blocking the connection is an adhesive seal.

25. A cold pack comprising:
an enclosure;
a solute within the enclosure;
a liquid within the enclosure; and
a membrane segregating the liquid from the solute;
wherein rupturing the membrane mixes the liquid with the solute to produce an endothermic solution within the enclosure; and
the enclosure includes a first compartment, a second compartment, and a connection between the first compartment and the second compartment, the connection adapted to pass the endothermic solution between the first and the second compartment; wherein
the liquid is water;
the solute is ammonium nitrate; and
the membrane is polyethylene.

26. The cold pack of claim 25 wherein each piece of the solute is substantially between 0.001 and 0.025 inches.

27. The cold pack of claim 25 wherein the solute is located in the first compartment and the liquid is located in the second compartment.

28. The cold pack of claim 25 wherein the solute is located in the first compartment and the liquid is located in the first compartment.
